(19) United States
(12) Patent Application Publication
(10) Pub. No.: US 2009/0143295 A1
(43) Pub. Date: Jun. 4, 2009

(54) PEPTIDE-BASED ANTIACNE REAGENTS

(75) Inventors: JOHN P. O'BRIEN, Oxford, PA (US); Hong Wang, Kennett Square, PA (US); Antoinette E. Wilkins, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/273,778

(22) Filed: Nov. 19, 2008

Related U.S. Application Data
(60) Provisional application No. 60/991,261, filed on Nov. 30, 2007.

(51) Int. Cl. A61K 47/48; A61K 31/60; A61K 31/085; A61K 38/00; A61K 38/10; A61K 38/08; A61P 17/10

(52) U.S. Cl. 514/12; 514/773; 514/162; 514/718; 514/2; 514/13; 514/14; 514/15

(57) ABSTRACT

Peptide-based antiacne reagents, formed by coupling a skin-binding peptide with an antiacne agent, are described. The skin-binding peptide portion of the peptide-based antiacne reagent binds strongly to the skin, thus keeping the antiacne agent coupled to the skin for a long lasting effect. Skin care compositions comprising the peptide-based antiacne reagents are also provided as well as a method of treating or preventing skin acne.

This application claims the benefit of U.S. Provisional Pat. App. No. 60/991,261, filed Nov. 30, 2007.

### FIELD OF THE INVENTION

The invention relates to the field of personal care products. More specifically, the invention relates to peptide-based antiacne reagents formed by coupling a skin-binding peptide with an antiacne agent.

### BACKGROUND OF THE INVENTION

Acne vulgaris, common acne, is a skin condition prevalent in adults, young adults and teenagers, affecting more than 50% of the U.S. population and 85% of all teenagers. Acne is characterized by mild to severe inflammatory lesions distributed over the face. The lesions may be distributed extensively or isolated to a minor facial area. The condition is caused by an overproduction of sebum and the colonization of clogged sebaceous gland ducts by Propionibacterium acnes (P. acnes), resulting in an inflammatory reaction. A more detailed description of the pathogenesis of acne can be found in Harry's Cosmeticology, 8^{th} Edition, Martin M. Rieger, Ed. Chemical Publishing, New York, N.Y., 2000, Chap. 22.

The primary treatment for acne is the topical application of agents that regenerate or slough off skin cells, absorb oil and/or reduce the level of P. acnes on the skin. Typically, the antiacne agent is applied to the skin as a component of a skin care composition. Examples of antiacne compositions are described U.S. Pat. App. Pub. No. 2004/0156873 to Gupta et al., EP App. Pub. No.1172087 A2 to Games et al., and U.S. Pat. App. Pub. No. 2006/0014834 to Vishnupad et al.

The problem with current acne treatment products is that they can be irritating to the skin, causing redness, dryness and chapping. In particular, keratolytic agents such as salicylic acid and benzoyl peroxide may cause severe irritation. Consumers are usually advised to start treatment with a lower concentration product and gradually increase the dosage to minimize irritation. In addition, the use of film-forming substances and oils in acne treatment compositions is avoided since these substances are know to promote the plugging of the sebaceous gland ducts. The compositions lack durability compared to other protective skin care products that contain high levels of oily ingredients such as sunscreens and night creams, for example. Consequently, a regimented schedule of reapplication is required to maintain efficacy. Additionally, acne compositions comprising peroxides can transfer to pillowcases, sheets, towels, clothing, etc., causing damage thereto due to bleaching. A non-irritating, long-lasting, durable acne treatment would represent an advance in the art.

In order to improve the durability of hair and skin care products, peptide-based hair conditioners, hair colorants, and other benefit agents have been developed (Huang et al., U.S. Pat. No. 7,220,405 and U.S. Pat. App. Pub. No. 2005/0226839). The peptide-based benefit agents are prepared by coupling a specific peptide sequence that has a high binding affinity to hair or skin with a benefit agent, such as a conditioner or colorant. The peptide portion binds to the hair or skin, thereby strongly coupling the benefit agent to the body surface. Additionally, U.S. Pat. No. 6,232,287 to Ruoslahti et al. describes the use of molecules that selectively home to various organs and tissues, such as skin, to deliver a therapeutic agent.

Peptides having a binding affinity to hair and skin have been identified using phage display screening techniques (Huang et al., supra; Estell et al. Int'l. App. Pub. No. 0179479; Murray et al., U.S. Pat. App. Pub. No. 2002/0098524; Janssen et al., U.S. Pat. App. Pub. No. 2003/0152976; and Janssen et al., Int'l. App. Pub. No. 04048399). Additionally, empirically-generated hair- and skin-binding peptides that are based on positively charged amino acids have been reported in Int'l. App. Pub. No. 2004/000257 to Rothe et al).

In view of the above, a need exists for antiacne agents that provide improved durability for long lasting effects and are easy and inexpensive to prepare.

The stated need has been addressed by designing peptide-based antiacne reagents that provide a long lasting effect by coupling skin-binding peptides, which bind to skin with high affinity, to antiacne agents.

### SUMMARY OF THE INVENTION

The invention provides peptide-based antiacne reagents formed by coupling at least one skin-binding peptide with at least one antiacne agent. Accordingly, in one embodiment the invention provides a peptide-based antiacne reagent having the general structure:

(SBPₘ)ₙ−(AA)_{y}, wherein
a) SBP is a skin-binding peptide;
b) AA is an antiacne agent;
c) m ranges from 1 to about 100;
d) n ranges from 1 to about 100; and
e) y ranges from 1 to about 100.

In another embodiment, the invention provides a peptide-based antiacne reagent having the general structure:

[(SBP)ₓ−Sₘ]ₙ−(AA)_{y}, wherein
a) SBP is a skin-binding peptide;
b) AA is an antiacne agent;
c) S is a spacer;
d) x ranges from 1 to about 10;
e) m ranges from 1 to about 100;
f) n ranges from 1 to about 100; and
g) y ranges from 1 to about 100.

In another embodiment, the invention provides skin care compositions comprising an effective amount of a peptide-based antiacne reagent.

Also provided are methods for treating or preventing acne comprising applying a skin care composition of the invention to the skin. In another embodiment, the invention provides a method for treating or preventing acne comprising the steps of:

a) providing a skin care composition comprising a peptide-based antiacne reagent selected from the group consisting of:

(SBPₘ)ₙ−(AA)_{y}; and i)

[(SBP)ₓ−Sₘ]ₙ−(AA)_{y} ii)

wherein
1) SBP is a skin-binding peptide;
2) AA is an antiacne agent;
3) n ranges from 1 to about 100;
4) S is a spacer;
5) m ranges from 1 to about 100;
6) x ranges from 1 to about 10; and
7) y ranges from 1 to about 100;

and wherein the skin-binding peptide is selected by a method comprising the steps of:
A) providing a combinatorial library of DNA associated peptides;
B) contacting the library of (A) with a skin sample to form a reaction solution comprising DNA associated peptide-skin complexes;
C) isolating the DNA associated peptide-skin complexes of (B);
D) amplifying the DNA encoding the peptide portion of the DNA associated peptide-skin complexes of (C); and
E) sequencing the amplified DNA of (d) encoding a skin-binding peptide, wherein the skin-binding peptide is identified; and

b) applying the skin care composition of (a) to the skin.

### Sequence Descriptions

The various embodiments of the invention can be more fully understood from the following detailed description and the accompanying sequence descriptions, which form a part of this application.

The following sequences conform with 37 C.F.R. 1.821-1.825 (“Requirements for Pat. Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs: 1-12 and 17-58 are the amino acid sequences of skin-binding peptides.

SEQ ID NO: 13 is the amino acid sequence of the protease Caspase 3 cleavage site.

SEQ ID NOs: 14-16 are the amino acid sequences of peptide spacers.

SEQ ID NOs: 18-22 and 45-58 are the amino acid sequences of skin care composition-resistant skin-binding peptides.

SEQ ID NOs: 59-87 are the amino acid sequences of antimicrobial peptides.

### DETAILED DESCRIPTION OF THE INVENTION

Peptide-based antiacne reagents are provided by coupling at least one skin-binding peptide to at least one antiacne agent, either directly or through a spacer. The peptide-based antiacne reagents may be used in skin care compositions to treat or prevent acne. The peptide-based antiacne reagents remain attached to the skin, through the affinity of the skin-binding peptide, thus providing a durable, long lasting effect. Alternatively, the antiacne agent may be encapsulated in a carrier or delivery agent such as a microsphere that can be coupled to the skin-binding peptide and be released over time. Due to the strong attachment of the peptide-based antiacne reagents to the skin, it may be possible to use lower concentrations of the reagents compared to conventional antiacne agents, thereby decreasing skin irritation.

The following definitions are used herein and should be referred to for interpretation of the claims and the specification.

As used herein, the indefinite articles “a” and “an” preceding an element or component of the invention are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof.

As used herein, the term “about” refers to modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein, the term “invention” or “present invention” is a non-limiting term and is not intended to refer to any single embodiment of the particular invention but encompasses all possible embodiments as described in the specification and the claims.

“SBP” means skin-binding peptide.

“AA” means antiacne agent.

“S” means spacer.

As used herein, the term “antiacne agent” or “anti-acne agent” refers to any chemical and/or biological antiacne agent (i.e. an antimicrobial peptide) that is effective in the treatment of acne and/or the symptoms associated therewith.

As used herein, the term “peptide” refers to two or more amino acids joined to each other by peptide bonds or modified peptide bonds.

As used herein, the term “skin-binding peptide” refers to peptide sequences that bind with high affinity to skin. The skin-binding peptides of the invention are from about 7 amino acids to about 60 amino acids, more preferably, from about 7 amino acids to about 35 amino acids, more preferably from about 7 to about 30 amino acids, and most preferably about 7 to about 20 amino acids in length.

As used herein, the term “DNA associated peptide” or “nucleic acid associated peptide” refers to a peptide having associated with it an identifying nucleic acid component. In the case of ribosome display or mRNA display, the DNA associated peptide may include peptides associated with their mRNA progenitor (i.e. an identifying nucleic acid component) that can be reverse translated into cDNA. In a phage display system, peptides are displayed on the surface of the phage while the DNA encoding the peptides is contained within the attached glycoprotein coat of the phage. The association of the coding DNA within the phage may be used to facilitate the amplification of the coding region for the identification of the peptide.

As used herein, the term “DNA associated peptide-skin complex” refers to a complex between skin and a DNA associated peptide wherein the peptide is bound to the skin via a binding site on the peptide.

As used herein, the term “skin” as used herein refers to human skin, or substitutes for human skin, such as pig skin, VITRO-SKIN® and EPIDERM™. Skin as a body surface will generally comprise a layer of epithelial cells and may additionally comprise a layer of endothelial cells.

As used herein, the term “skin surface” will mean the surface of skin that may serve as a substrate for the binding of a skin-binding peptide and/or a peptide-based antiacne reagent.

As used herein, the terms “coupling” and “coupled” refer to any chemical association and includes both covalent and non-covalent interactions. In one embodiment, the term “coupling” or “coupled” refers to a non-covalent interaction. In another embodiment, the term “coupling” or “coupled” refers to a covalent interaction.

As used herein, the term “MB₅₀” refers to the concentration of the binding peptide that gives a signal that is 50% of the maximum signal obtained in an ELISA-based binding assay (see Example 3 of U.S. Pat. App. Pub. 2005/022683, incorporated herein by reference). The MB₅₀ provides an indication of the strength of the binding interaction or affinity of the components of the complex. The lower the value of MB₅₀, the stronger the interaction of the peptide with its corresponding substrate.

As used herein, the terms “binding affinity” or “affinity” refers to the strength of the interaction of a binding peptide with its respective substrate. The binding affinity may be reported in terms of the MB₅₀ value as determined in an ELISA-based binding assay or as a K_{D} (equilibrium dissociation constant) value, which may be deduced using surface plasmon resonance (SPR).

As used herein, the term “strong affinity” refers to a binding affinity, as measured as an MB₅₀ or K_{D} value, of 10⁻⁴ M, preferably 10⁻⁵ M or less, preferably less than 10⁻⁶ M, more preferably less than 10⁻⁷ M, more preferably less than 10⁻⁸ M, even more preferably less than 10⁻⁹ M, and most preferably less than 10⁻¹⁰ M. The lower the value of MB₅₀ or K_{D}, the stronger affinity of the peptide interacting with its corresponding substrate.

As used herein, the term “stringency” as it is applied to the selection of the skin-binding peptides of the present invention, refers to the concentration of the eluting agent (usually a detergent) used to elute peptides from the skin. Higher concentrations of the eluting agent provide more stringent conditions.

As used herein, the term “amino acid” refers to the basic chemical structural unit of a protein or polypeptide.

As used herein, “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

As used herein, “synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

As used herein, “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “expression” refers to the transcription and stable accumulation of sense (mRNA) or antisense RNA derived from the nucleic acid fragment of the invention. Expression may also refer to translation of mRNA into a polypeptide.

As used herein, the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

As used herein, the term “host cell” refers to a cell which has been transformed or transfected, or is capable of transformation or transfection by an exogenous polynucleotide sequence.

As used herein, the terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

As used herein, the term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

As used herein, the term “peptide-based” refers to an interfacial material comprised of a compound pertaining to or having the nature or the composition of the peptide class. Interfacial refers to the quality of the peptide-based material described herein as connecting one material to another.

As used herein, “PCR” or “polymerase chain reaction” is a technique used for the amplification of specific DNA segments (U.S. Pat. Nos. 4,683,195 and 4,800,159).

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| (or as defined herein) | | |

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Skin-Binding Peptides

Skin-binding peptides (SBP), as defined herein, are peptide sequences that bind with high affinity to skin. The skin-binding peptides of the invention are from about 7 amino acids to about 60 amino acids, more preferably, from about 7 amino acids to about 35 amino acids, more preferably about 7 to about 30 amino acids, and most preferably from about 7 to about 20 amino acids in length. Suitable skin-binding peptides may be selected using methods that are well known in the art or may be generated empirically.

The skin-binding peptides may be generated randomly and then selected against a specific skin sample based upon their binding affinity for skin, as described by Huang et al. U.S. Pat. No. 7,220,405. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500); ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate such biological peptide libraries are also described in Dani, M., J. of Receptor & Signal Transduction Res., 21 (4):447-468 (2001), Sidhu et al., Methods in Enzymology 328:333-363 (2000), Kay et al., Combinatorial Chemistry & High Throughput Screening, Vol. 8:545-551 (2005), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England Biolabs (Beverly, Mass.).

A preferred method to randomly generate peptides is by phage display. Since its introduction in 1985, phage display has been widely used to discover a variety of ligands including peptides, proteins and small molecules for drug targets (Dixit, J. of Sci. & Ind. Research, 57:173-183 (1998)). The applications have expanded to other areas such as studying protein folding, novel catalytic activities, DNA-binding proteins with novel specificities, and novel peptide-based biomaterial scaffolds for tissue engineering (Hoess, Chem. Rev. 101:3205-3218 (2001) and Holmes, Trends Biotechnol. 20:16-21 (2002)). Whaley et al. (Nature 405:665-668 (2000)) disclose the use of phage display screening to identify peptide sequences that can bind specifically to different crystallographic forms of inorganic semiconductor substrates.

A modified screening method that comprises contacting a peptide library with an anti-target to remove peptides that bind to the anti-target, then contacting the non-binding peptides with the target has been described (Estell et al. in Int'l. App. Pub. No. 01/79479, Murray etal. in U.S. Pat. App. Pub. No. 2002/0098524, and Janssen et al. in U.S. Pat. App. Pub. No. 2003/0152976). Using that method, a peptide binds to hair and not to skin and a peptide that binds to skin and not hair were identified. Using the same method, Janssen et al. in Int'l. App. Pub. No. 04/048399 identified other skin-binding and hair-binding peptides, as well as several other binding motifs.

Phage display is a selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. As used herein, “biopanning” may be used to describe any selection procedure (phage display, ribosome display, mRNA-display, etc.) where a library of displayed peptides a library of displayed peptides is panned against a specified target material (e.g. hair). In its simplest form, phage display biopanning is carried out by incubating the pool of phage-displayed variants with a target of surface interest (the target material is often immobilized on a plate or bead), washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

The skin-binding peptides may be identified using the following process. A suitable library of phage-peptides is generated using the methods described above or the library is purchased from a commercial supplier. After the library of phage-peptides has been generated, the library is contacted with an appropriate amount of skin sample to form a reaction solution. Human skin samples may be obtained from cadavers or in vitro human skin cultures. Additionally, pig skin, VITRO-SKIN® (available from IMS inc., Milford, Conn.) and EPIDERM™ (available from Mattek corp., Ashland, Mass.) may be used as substitutes for human skin. The library of phage-peptides is dissolved in a suitable solution for contacting the skin substrate. The test substrate may be suspended in the solution or may be immobilized on a plate or bead. A preferred solution is a buffered aqueous saline solution containing a surfactant. A suitable solution is Tris-buffered saline (TBS) with 0.05 to 0.5% TWEEN® 20. The solution may additionally be agitated by any means in order to increase the mass transfer rate of the peptides to the substrate, thereby shortening the time required to attain maximum binding.

Upon contact, a number of the randomly generated phage-peptides will bind to the substrate to form a phage-peptide-substrate complex. Unbound phage-peptide may be removed by washing. After all unbound material is removed, phage-peptides having varying degrees of binding affinities for the substrate may be fractionated by selected washings in buffers having varying stringencies. Increasing the stringency of the buffer used increases the required strength of the bond between the phage-peptide and substrate in the phage-peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that phage-peptides having increasing binding affinities for the substrate may be eluted by repeating the selection process using buffers with increasing stringencies. The eluted phage-peptides can be identified and sequenced by any means known in the art. For example, the eluted DNA associated peptides and the remaining bound DNA associated peptides may be amplified by infecting/transfecting a bacterial host cell, such as E. coli ER2738, as described by Huang et al. in U.S. Pat. No. 7,220,405. The infected host cells are grown in a suitable growth medium, such as LB (Luria-Bertani) medium, and this culture is spread onto agar, containing a suitable growth medium, such as LB medium with IPTG (isopropyl β-D-thiogalactopyranoside) and S-Gal™ (3,4-cyclohexenoesculetin-β-D-galactopyranoside). After growth, the plaques are picked for DNA isolation and sequencing to identify the skin-binding peptide sequences. Alternatively, the eluted DNA associated peptides and the remaining bound DNA associated peptides may be amplified using a nucleic acid amplification method, such as the polymerase chain reaction (PCR), to amplify the DNA comprising the peptide coding region. In that approach, PCR is carried out on the DNA encoding the eluted DNA associated peptides and/or the remaining bound DNA associated peptides using the appropriate primers, as described by Janssen et al. in U.S. Pat. App. Pub. No. 2003/0152976.

In one embodiment, the eluted DNA associated peptides and the remaining bound DNA associated peptides are amplified by infecting a bacterial host cell as described above, the amplified DNA associated peptides are contacted with a fresh skin sample, and the entire process described above is repeated one or more times to obtain a population that is enriched in skin-binding DNA associated peptides. After the desired number of biopanning cycles, the amplified DNA associated peptide sequences are determined using standard DNA sequencing techniques that are well known in the art to identify the skin-binding peptide sequences. Skin-binding peptide sequences identified using this method include, but are not limited to, SEQ ID NO:1, 7-12, 17, and 23-44.

Skin-binding peptides that are resistant to skin care compositions, as described by Wang et al. in co-pending and commonly owned U.S. patent application Ser. No. 11/359162 (published as U.S. 2006/0199206), may also be used in the peptide-based antidandruff reagents of the invention. Examples of skin care composition-resistant skin-binding peptides include, but are not limited to, the peptide sequences given as SEQ ID NOs:18-22 and 45-58.

In one embodiment, the skin-binding peptide is selected from the group consisting of SEQ ID NOs:1, 2, 3,4, 5, 6, 7, 8, 9, 10, 11, 12, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, and 58.

Additionally, skin-binding peptides that are resistant to shampoo and other hair care compositions, such as conditioners, may be identified using variations of the methods described by O'Brien et al. in U.S. Pat. App. Pub. No. 2006/0073111, Wang et al. in U.S. Pat. App. Pub. No. U.S. 2007/0196305, and Wang et al. in U.S. Pat. App. Pub. No. 2006/0199206 for identifying shampoo resistant hair-binding peptides, hair conditioner-resistant hair-binding peptides, and skin care composition-resistant skin-binding peptides, respectively. Briefly, the DNA associated peptide-skin complex is contacted with the desired composition (e.g. a shampoo or hair conditioner) at least one time in the biopanning process described above. For example, the phage peptide library may be dissolved in the hair care composition which is then contacted with the skin sample. Alternatively, the DNA associated peptide-skin complex, formed by contacting the skin sample with the phage display library, may be subsequently contacted with a hair care composition. Additionally, any combination of these hair care composition-contacting methods may be used.

Skin-binding peptide sequences may also be determined using the method described by Lowe, D. in U.S. Pat. App. Pub. No. 2006/0286047. The Lowe method provides a means for determining the sequence of a peptide binding motif having affinity for a particular substrate, for example skin. First, a population of binding peptides for the substrate of interest is identified by biopanning using a combinatorial method, such as phage display. Rather than using many rounds of biopanning to identify specific binding peptide sequences and then using standard pattern recognition techniques to identify binding motifs, as is conventionally done in the art, the method requires only a few rounds of biopanning. The sequences in the population of binding peptides, which are generated by biopanning, are analyzed by identifying subsequences of 2, 3, 4, and 5 amino acid residues that occur more frequently than expected by random chance. The identified subsequences are then matched head to tail to give peptide motifs with substrate binding properties. This procedure may be repeated many times to generate long peptide sequences.

Alternatively, skin-binding peptide sequences may be generated empirically by designing peptides that comprise positively charged amino acids, which can bind to skin via electrostatic interaction, as described by Rothe et al. in Int'l. App. Pub. No. 2004/000257. The empirically generated skin-binding peptides have between about 7 amino acids to about 60 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically generated skin-binding peptides include, but are not limited to, SEQ ID NOs: 2, 3, 4, 5, and 6.

The skin-binding peptide may further comprise at least one cysteine or lysine residue on at least one of the C-terminal end or the N-terminal end of the skin-binding peptide sequence to facilitate coupling with the antidandruff agent, as described below. Examples of a skin-binding peptide having a lysine residue on the C-terminal end of the binding sequence are given as SEQ ID NO:17 and SEQ ID NO: 42. Additionally, the skin-binding peptide may further comprise at least one proline or aspartic acid residue on at least one of the C-terminal end or the N-terminal end of the skin-binding peptide sequence. The terminal aspartic acid (D) or proline (P) residues may result from the use of acid-labile DP cleavage sites in the biological production of the peptides. Examples of various skin-binding peptides are provided below in Table A.

TABLE A
Examples of Skin-Binding Peptides

| | | | |
|---|---|---|---|
| Hair and | KRGRHKRPKRHK | 2 | US 2007-0065387 |
| skin | | | US 2007-0110686 |
| (Empirical) | | | US 2007-0067924 |
| Hair and | RLLRLLR | 3 | US 2007-0065387 |
| skin | | | US 2007-0110686 |
| Empirical) | | | |
| Hair and | HKPRGGRKKALH | 4 | US 2007-0065387 |
| skin | | | US 2007-0110686 |
| Empirical) | | | |
| Hair and | KPRPPHGKKHRPKHRPKK | 5 | US 2007-0065387 |
| skin | | | US 2007-0110686 |
| Empirical) | | | |
| Hair and | RGRPKKGHGKRPGHRARK | 6 | US 2007-0065387 |
| skin | | | US 2007-0110686 |
| Empirical) | | | |
| Skin | TPFHSPENAPGS | 1 | US 11/877,692 |
| | | | US 2005-0249682 |
| Skin | TPFHSPENAPGSK | 17 | US 2007-0110686 |
| Skin | TPFHSPENAPGSGGGS | 23 | US 2007-0110686 |
| Skin | TPFHSPENAPGSGGGSS | 24 | US 2007-0110686 |
| Skin | TPFHSPENAPGSGGG | 25 | US 2007-0110686 |
| Skin | FTQSLPR | 26 | US 11/877,692 |
| | | | US 2005-0249682 |
| Skin | KQATFPPNPTAY | 7 | US 11/877,692 |
| | | | US 2005-0249682 |
| | | | WO2004048399 |
| Skin | HGHMVSTSQLSI | 8 | US 11/877,692 |
| | | | US 2005-0249682 |
| | | | WO2004048399 |
| Skin | LSPSRMK | 9 | US 11/877,692 |
| | | | US 2005-0249682 |
| | | | WO2004048399 |
| Skin | LPIPRMK | 10 | US 2005-0249682 |
| | | | WO2004048399 |
| Skin | HQRPYLT | 11 | US 2005-0249682 |
| | | | WO2004048399 |
| Skin | FPPLLRL | 12 | US 2005-0249682 |
| | | | WO2004048399 |
| Skin | QATFMYN | 27 | WO2004048399 |
| Skin | VLTSQLPNHSM | 28 | WO2004048399 |
| Skin | HSTAYLT | 29 | WO2004048399 |
| Skin | APQQRPMKTFNT | 30 | WO2004048399 |
| Skin | APQQRPMKTVQY | 31 | WO2004048399 |
| Skin | PPWLDLL | 32 | WO2004048399 |
| Skin | PPWTFPL | 33 | WO2004048399 |
| Skin | SVTHLTS | 34 | WO2004048399 |
| Skin | VITRLTS | 35 | WO2004048399 |
| Skin | DLKPPLLALSKV | 36 | WO2004048399 |
| Skin | SHPSGALQEGTF | 37 | WO2004048399 |
| Skin | FPLTSKPSGACT | 38 | WO2004048399 |
| Skin | DLKPPLLALSKV | 39 | WO2004048399 |
| Skin | PLLALHS | 40 | WO2004048399 |
| Skin | VPISTQI | 41 | WO2004048399 |
| Skin | YAKQHYPISTFK | 42 | WO2004048399 |
| Skin | HSTAYLT | 43 | WO2004048399 |
| Skin | STAYLVAMSAAP | 44 | WO2004048399 |
| Skin (Body | SVSVGMKPSPRP | 19 | US 11/877,692 |
| Wash | | | US 2006-0199206 |
| Resistant) | | | |
| Skin (Body | TMGFTAPRFPHY | 18 | US 11/877,692 |
| Wash | | | US 2006-0199206 |
| Resistant) | | | |
| Skin (Body | KTMGFTAPRFPHY | 22 | US 11/877,692 |
| Wash | | | US 2006-0199206 |
| Resistant) | | | |
| Skin (Body | NLQHSVGTSPVW | 45 | US 11/877,692 |
| Wash | | | US 2006-0199206 |
| Resistant) | | | |
| Skin (Body | QLSYHAYPQANHHAP | 20 | US 11/877,692 |
| Wash | | | US 2006-0199206 |
| Resistant) | | | |
| Skin (Body | NQAASITKRVPY | 46 | US 2006-0199206 |
| Wash | | | |
| Resistant) | | | |
| Skin (Body | SGCHLVYDNGFCDH | 21 | US 11/877,692 |
| Wash | | | US 2006-0199206 |
| Resistant) | | | |
| Skin (Body | ASCPSASHADPCAH | 47 | US 11/877,692 |
| Wash | | | US 2006-0199206 |
| Resistant) | | | |
| Skin (Body | NLCDSARDSPRCKV | 48 | US 11/877,692 |
| Wash | | | US 2006-0199206 |
| Resistant) | | | |
| Skin (Body | NHSNWKTAADFL | 49 | US 11/877,692 |
| Wash | | | US 2006-0199206 |
| Resistant) | | | |
| Skin (Body | GSSTVGRPLSYE | 50 | US 2006-0199206 |
| Wash | | | |
| Resistant) | | | |
| Skin (Body | SDTISRLHVSMT | 51 | US 11/877,692 |
| Wash | | | US 2006-0199206 |
| Resistant) | | | |
| Skin (Body | SPLTVPYERKLL | 52 | US 2006-0199206 |
| Wash | | | |
| Resistant) | | | |
| Skin (Body | SPYPSWSTPAGR | 53 | US 11/877,692 |
| Wash | | | US 2006-0199206 |
| Resistant) | | | |
| Skin (Body | VQPITNTRYEGG | 54 | US 2006-0199206 |
| Wash | | | |
| Resistant) | | | |
| Skin (Body | WPMHPEKGSRWS | 55 | US 2006-0199206 |
| Wash | | | |
| Resistant) | | | |
| Skin (Body | DACSGNGHPNNCDR | 56 | US 11/877,692 |
| Wash | | | US 2006-0199206 |
| Resistant) | | | |
| Skin (Body | DHCLGRQLQPVCYP | 57 | US 2006-0199206 |
| Wash | | | |
| Resistant) | | | |
| Skin (Body | DWCDTIIPGRTCHG | 58 | US 11/877,692 |
| Wash | | | US 2006-0199206 |
| Resistant) | | | |

### Binding Affinity

In one embodiment, the skin-binding peptides used in the present peptide-based antidandruff reagents exhibit a strong affinity for skin. The affinity of the peptide for the skin can be expressed in terms of the dissociation constant K_{D}. K_{D} (expressed as molar concentration) corresponds to the concentration of peptide at which the binding site on the target is half occupied, i.e. when the concentration of target with peptide bound (bound target material) equals the concentration of target with no peptide bound. The smaller the dissociation constant, the more tightly bound the peptide is; for example, a peptide with a nanomolar (nM) dissociation constant binds more tightly than a peptide with a micromolar (μM) dissociation constant. In one embodiment, the skin-binding peptides have a K_{D} of 10⁻⁴ M or less, preferably 10⁻⁵ M or less, more preferably 10⁻⁶ M or less, even more preferably 10⁻⁷ M or less, yet even more preferably 10⁻⁸ M or less, and most preferably 10⁻⁹ M or less.

Alternatively, one of skill in the art can also use an ELISA-based assay to calculate a relative affinity of the peptide for the target material (reported as an “MB₅₀” value; see Example 3 of U.S. Pat. App. Pub. 2005/022683; incorporated herein by reference). As used herein, the term “MB₅₀” refers to the concentration of the binding peptide that gives a signal that is 50% of the maximum signal obtained in an ELISA-based binding assay. The MB₅₀ provides an indication of the strength of the binding interaction or affinity of the components of the complex. The lower the value of MB₅₀, the stronger the interaction of the peptide with its corresponding substrate. In one embodiment, the MB₅₀ value (reported in terms of molar concentration) for the skin-binding peptide is 10⁻⁴ M or less, preferably 10⁻⁵ M or less, more preferably 10⁻⁶ M or less, even more preferably 10⁻⁷ M or less, and most preferably 10⁻⁸ M or less.

### Production of Skin-Binding Peptides

The skin-binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the skin-binding peptides may be produced in heterologous host cells, particularly in the cells of microbial hosts, as described by Huang et al. in U.S. Pat. No. 7,220,405.

Preferred heterologous host cells for expression of the skin-binding peptides are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides described herein. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the skin-binding peptides or peptide-based reagents described herein. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the peptides described herein including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence is typically employed to transform an appropriate host to permit the host to express the peptide of the present invention. Cell-free translation systems can also be employed to produce such peptides using RNAs derived from the DNA constructs of the present invention. Optionally it may be desired to produce the gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP Pat. No.546049 and Int'l. App. Pub. No. 9324631). The secretion signal DNA or facilitator may be located between the expression-controlling DNA and the instant gene or gene fragment, and in the same reading frame with the latter.

### Peptide-Based Antiacne Reagents

The peptide-based antiacne reagents of the present invention are formed by coupling at least one skin-binding peptide (SBP) with at least one antiacne agent (AA). The skin-binding peptide part of the antiacne reagent binds strongly to the skin, thus keeping the antiacne agent attached to the skin for a long lasting effect. Suitable skin-binding peptides include, but are not limited to, the skin binding peptides described above. It may also be desirable to link two or more skin-binding peptides together, either directly or through a spacer, to enhance the interaction with the skin. Methods to prepare these multiple skin-binding peptides and suitable spacers are described below.

Antiacne agent, as herein defined, refers to any chemical that is effective in the treatment of acne and/or the symptoms associated therewith. Antiacne agents are well known in the art such as U.S. Pat. App. Pub. No. 2006/0008538 to Wu et al. (in particular, paragraph 0014) and U.S. Pat. No. 5,607,980 to McAtee et al., (in particular, column 11, lines 10-25) both of which are incorporated herein by reference. Examples of useful antiacne agents include, but are not limited to keratolytics, such as salicylic acid, derivatives of salicylic acid, and resorcinol; retinoids, such as retinoic acid, tretinoin, adapalene, tazarotene,; sulfur-containing D- and L-amino acids and their derivatives and salts; lipoic acid; antibiotics and antimicrobials, such as benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, and meclocycline; sebostats, such as flavonoids; and bile salts, such as scymnol sulfate and its derivatives, deoxycholate, and cholate; and combinations thereof. These agents are well known and commonly used in the field of personal care.

Additionally, the antiacne agent may be an antimicrobial peptide having activity against P. acnes. Antimicrobial peptides are ubiquitous in nature and play an important role in the innate immune system of many species (Zasloff, Nature 415:389-395 (2002); Epand et al., Biochim Biophys Acta 1462:11-28 (1999)). The antimicrobial peptide may be a naturally occurring peptide or an analog thereof, or it may be a synthetic peptide. As used herein an “analog” refers to a naturally-occurring antimicrobial peptide that has been chemically modified to improve its effectiveness and/or reduce its toxic side effects. The antimicrobial peptide may be a peptide known to be effective against Gram positive bacteria. Non-limiting examples include lantibiotics, such as nisin, subtilin, epidermin and gallidermin; defensins; attacins, such as sarcotoxin; cecropins, such as cecropin A, bactericidin, and lepidopteran; magainins; melittins; histatins; brevinins; and combinations thereof. Additionally, antimicrobial peptides having activity against P. acnes have been reported, for example, in U.S. Pat. App. Pub. No. 2005/0282755 to Hart et al., U.S. Pat. App. Pub. No. 2005/02455452 to Hogenhaug, U.S. Pat. App. Pub. No.2005/0209157 to Owen, and U.S. Pat. No. 6,255,279 to Christophers et al. Suitable examples of antimicrobial peptides having reported activity against P. acnes include, but are not limited to, novispirins (Hogenhaug, supra), and those given by SEQ ID NOs:59-87 as shown in Table B below (see U.S. Pat. App. Pub. No.2007/0265431). These antimicrobial peptides may be prepared using the methods described above for the preparation of skin-binding peptides.

TABLE B
Antimicrobial active peptide sequences.

| Species | SEQ | |
|---|---|---|
| of origin | ID NO. | Sequence |
| Artificial | 59 | PKGLKKLLKGLKKLLKL |
| Artificial | 60 | KGLKKLLKGLKKLLKL |
| Artificial | 61 | KGLKKLLKLLKKLLKL |
| Artificial | 62 | LKKLLKLLKKLLKL |
| Artificial | 63 | LKKLLKLLKKLL |
| Artificial | 64 | VAKKLAKLAKKLAKLAL |
| Artificial | 65 | FAKLLAKALKKLL |
| Artificial | 66 | KGLKKGLKLLKKLLKL |
| Artificial | 67 | KGLKKLLKLGKKLLKL |
| Artificial | 68 | KGLKKLGKLLKKLLKL |
| Artificial | 69 | KGLKKLLKLLKKGLKL |
| Artificial | 70 | KGLKKLLKLLKKLGKL |
| Artificial | 71 | FALALKALKKLKKALKKAL |
| Artificial | 72 | FAKKLAKLAKKLAKLAL |
| Artificial | 73 | FAKLLAKLAKKLL |
| Artificial | 74 | FAKKLAKLALKLAKL |
| Artificial | 75 | FAKKLAKKLL |
| Artificial | 76 | FAKLLAKLAKKVL |
| Artificial | 77 | KYKKALKKLAKLL |
| Artificial | 78 | FALLKALLKKAL |
| Artificial | 79 | KRLFKKLKFSLRKY |
| Artificial | 80 | KRLFKKLLFSLRKY |
| Artificial | 81 | LLLFLLKKRKKRKY |
| H. cecropia | 82 | KWKLFKKIEKVGQNIRDGIIKAGPAVAWGQAT |
| | | QIAK |
| Xenopus | 83 | GIGKFLHSAKKFGKAFVGEIMNS |
| Xenopus | 84 | GIGKFLKKAKKFGKAFVKILKK |
| Bos Taurus | 85 | RLCRIWIRVCR |
| Bos Sp. | 86 | ILPWKWPWWPWRR |
| H. sapiens | 87 | DSHAKRHHGYKRKFHEKHHSHRGY |

The peptide-based antiacne reagents are prepared by coupling at least one specific skin-binding peptide to at least one antiacne agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based antiacne reagent may be prepared by mixing the peptide with the antiacne agent and the optional spacer (if used) and allowing sufficient time for the interaction to occur. The unbound materials may be separated from the resulting peptide-based antiacne reagent using methods known in the art, for example, gel permeation chromatography.

The peptide-based antiacne reagents of the invention may also be prepared by covalently attaching at least one specific skin-binding peptide to at least one antiacne agent, either directly or through a spacer. Any known peptide or protein conjugation chemistry may be used to form the peptide-based antiacne reagents of the present invention. Conjugation chemistries are well-known in the art (see for example, G. T. Hermanson, Bioconiugate Techniques, 2^{nd} Ed., Academic Press, New York (2008)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based antiacne reagent. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, isocyanate, epoxide, or aldehyde groups on the antiacne agent for coupling to the skin-binding peptide. These modifications may be done using routine chemistry such as oxidation, reduction, phosgenation, and the like, which is well known in the art.

It may also be desirable to couple the skin-binding peptide to the antiacne agent via a spacer. The spacer serves to separate the antiacne agent from the peptide to ensure that the agent does not interfere with the binding of the peptide to the skin. The spacer may be any of a variety of molecules, such as alkyl chains, phenyl compounds, ethylene glycol, amides, esters and the like. Preferred spacers have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, and ethyl, propyl, hexyl, steryl, cetyl, and palmitoyl alkyl chains. The spacer may be covalently attached to the peptide and the antiacne agent using any of the coupling chemistries described above. In order to facilitate incorporation of the spacer, a bifunctional cross-linking agent that contains a spacer and reactive groups at both ends for coupling to the peptide and the antiacne agent may be used.

Additionally, the spacer may be a peptide comprising any amino acid and mixtures thereof. The preferred peptide spacers are comprised of the amino acids proline, lysine, glycine, alanine, and serine, and mixtures thereof. In addition, the peptide spacer may comprise a specific enzyme cleavage site, such as the protease Caspase 3 site, given as SEQ ID NO:13, which allows for the enzymatic removal of the antiacne agent from the skin. The peptide spacer may be from 1 to about 50 amino acids, preferably from 1 to about 20 amino acids in length. Exemplary peptide spacers comprise amino acid sequences including, but are not limited to, SEQ ID NOs: 14, 15, and 16. These peptide spacers may be linked to the binding peptide sequence by any method known in the art. For example, the entire binding peptide-peptide spacer diblock may be prepared using the standard peptide synthesis methods described above. In addition, the binding peptide and peptide spacer blocks may be combined using carbodiimide coupling agents (Hermanson, supra), diacid chlorides, diisocyanates and other difunctional coupling reagents that are reactive to terminal amine and/or carboxylic acid groups on the peptides. Alternatively, the entire binding peptide-peptide spacer diblock may be prepared using the recombinant DNA and molecular cloning techniques described above. The spacer may also be a combination of a peptide spacer and an organic spacer molecule, which may be prepared using the methods described above.

In the embodiment wherein the antiacne agent is an antimicrobial peptide, the skin-binding peptide may be coupled to the antimicrobial peptide, with or without a spacer, using the methods described above. For example, the entire skin binding peptide-antimicrobial peptide diblock or the skin binding peptide-peptide spacer-antimicrobial peptide triblock may be prepared using the standard peptide synthesis methods described above. In addition, the skin binding peptide, the optional peptide spacer, and the antimicrobial peptide blocks may be combined using coupling agents, as described above. Alternatively, the entire skin binding peptide-optional peptide spacer-antimicrobial peptide diblock or triblock may be prepared using the recombinant DNA and molecular cloning techniques described above.

It may also be desirable to have multiple skin-binding peptides coupled to the antiacne agent to enhance the interaction between the peptide-based antiacne reagent and the skin. Either multiple copies of the same skin-binding peptide or a combination of different skin-binding peptides may be used. Typically, 1 to about 100 skin-binding peptides can be coupled to an antiacne agent. Additionally, multiple peptide sequences may be linked together and attached to the antiacne agent, as described above. Typically, up to about 100 skin-binding peptides may be linked together. Moreover, multiple antiacne agents (AA) may be coupled to the skin-binding peptide. Therefore, in one embodiment of the present invention, the peptide-based antiacne reagents are compositions consisting of a skin-binding peptide (SBP) and an antiacne agent (AA), having the general structure (SBPₘ)ₙ−(AA)_{y}, where m, n and y independently range from 1 to about 100, preferably from 1 to about 10.

In another embodiment, the peptide-based antiacne reagents contain a spacer (S) separating the skin-binding peptide from the antiacne agent, as described above. Multiple copies of the skin-binding peptide may be coupled to a single spacer molecule. Additionally, multiple copies of the peptides may be linked together via spacers and coupled to the antiacne agent via a spacer. Moreover, multiple antiacne agents (AA) may be coupled to the spacer. In this embodiment, the peptide-based antiacne reagents are compositions consisting of a skin-binding peptide, a spacer, and an antiacne agent, having the general structure [(SBP)ₓ−Sₘ]ₙ−(AA)_{y}, where x ranges from 1 to about 10, preferably x is 1, and m, n and y independently range from 1 to about 100, preferably from 1 to about 10.

It should be understood that as used herein, SBP is a generic designation and is not meant to refer to a single skin-binding peptide sequence. Where m, n or x as used above, is greater than 1, it is well within the scope of the invention to provide for the situation where a series of skin-binding peptides of different sequences may form a part of the composition. It should also be understood that as used herein, AA is a generic term and is not meant to refer to a single antiacne agent. Where y as used above, is greater than 1, it is well within the scope of the invention to provide for the situation where a number of different antiacne agents may form a part of the composition. Additionally, it should be understood that these structures do not necessarily represent a covalent bond between the peptide, the antiacne agent, and the optional spacer. As described above, the coupling interaction between the peptide, the antiacne agent, and the optional spacer may be either covalent or non-covalent.

### Skin Care Compositions

The peptide-based antiacne reagents of the invention may be used in skin care compositions to treat or prevent acne. Skin care compositions are herein defined as compositions for the treatment of skin including, but not limited to, skin conditioners, moisturizers, foundations, anti-wrinkle products, skin cleansers, and body washes. The skin care compositions of the present invention include any composition that may be topically applied to the skin, including but not limited to, lotions, creams, gels, sticks, sprays, ointments, cleansing liquid washes, cleansing solid bars, pastes, foams, powders, shaving creams, and wipes.

The skin care compositions of the invention may comprise several types of cosmetically-acceptable topical carriers including, but not limited to solutions, colloidal suspensions, dispersions, emulsions (microemulsions, nanoemulsions, multiple and non-aqueous emulsions), hydrogels, and vesicles (liposomes, niosomes, novasomes). Components and formulation methods of suitable cosmetically-acceptable topical carriers are well known in the art and are described for example by U.S. Pat. No. 6,797,697 to Sieberg et al., U.S. Pat. App. Pub. No. 2005/0142094 to Kumar, U.S. Pat. App. Pub. No. 2005/0008604 to Schultz et al., Int'l. App. Pub. No. 2006/029818 to Beumer et al., and Int'l. App. Pub. No. 2000/062743 to Robinson et al. Those skilled in the art will appreciate the various methods for producing these various product forms.

The skin care compositions of the present invention comprise an effective amount of at least one peptide-based antiacne reagent, ranging from about 0.001% to about 10%, preferably from about 0.1% to about 5%, and more preferably from about 0.5% to about 3% by weight relative to the total weight of the composition. As used here, the term “effective amount” is that amount of the peptide-based antiacne reagent in the skin care composition necessary to achieve the desired improvement.

Typically, the cosmetically acceptable medium for skin care compositions comprises water and other solvents which include, but are not limited to, mineral oils and fatty alcohols. The cosmetically-acceptable medium is from about 10% to about 99.99% by weight of the composition, preferably from about 50% to about 99% by weight of the composition, and can, in the absence of other additives, form the balance of the composition.

The skin care composition may further comprise the following basic cosmetic raw materials, including, but not limited to hydrocarbons, esters, fatty alcohols, fatty acids, emulsifying agents, humectants, viscosity modifiers, and silicone-based materials. The compositions of the present invention may contain a wide range of these basic components. The total concentration of added ingredients usually is less than 50%, preferably less than 20%, and most preferably less than 10% by weight of the total composition. Those skilled in the art will appreciate the various concentrations and combinations for employing these basic components to achieve the desired product form.

Suitable hydrocarbons which may be used in the compositions of the invention include, but are not limited to mineral oil, isohexadecane, squalane, hydrogenated polyisobutene, petrolatum, paraffin, microcrystalline wax, and polyethylene.

Suitable esters which may be used in the compositions of the invention include, but are not limited to isopropyl palmitate, octyl stearate, caprylic/capric triglyceride, plant waxes (Canelilla, Caranauba), vegetable oils (natural glycerides) and plant oils (Jojoba).

Suitable fatty alcohols which may be used in the compositions of the invention include, but are not limited to myristyl, cety, stearyl, isostearyl, and behenyl.

Suitable emulsifying agents which may be used in the compositions of the invention include, but are not limited to anionic (TEA/K stearate (triethanolamine/potassium stearate), sodium lauryl stearate, sodium cetearyl sulfate, and beeswax/Borax), nonionic (glycerol di-stearate, PEG (polyethyleneglycol)-100 Stearate, Polysorbate 20, steareth 2 and steareth 20), and cationic (distearyldimethylammonium chloride, behenalkonium chloride and steapyrium chloride), polymeric (acrylates/C10-30 alkyl acrylate crosspolymer, polyacrylamide, polyquaternium-37, propylene glycol, dicaprylate/dicaparate and PPG-1 Trideceth-6), and silicone-based materials (alkyl modified dimethicone copolyols), and polyglyceryl esters, and ethoxylated di-fatty esters.

Exemplary humectants for use in the compositions of the invention include, but are not limited to propylene glycol, sorbitol, butylene glycol, hexylene glycol, acetamide MEA (acetylethanolamine), honey, and sodium PCA (sodium-2-pyrrolidone carboxylate).

Viscosity modifiers which may be used in the compositions of the invention include, but are not limited to xanthum gum, magnesium aluminum silicate, cellulose gum, and hydrogenated castor oil.

Further, the skin care compositions may comprise one or more conventional functional cosmetic or dermatological additives or adjuvants, providing that they do not interfere with the mildness, performance or aesthetic characteristics desired in the final products. The CTFA (The Cosmetic, Toiletry, and Fragrance Association; now known as the Personal Care Products Council) International Cosmetic Ingredient Dictionary and Handbook, Eleventh Edition (2006), and McCutcheon's Functional Materials, North America and Internationals Editions, MC Publishing Co. (2007) describe a wide variety of cosmetic and pharmaceutical ingredients commonly used in skin care compositions, which are suitable for use in the compositions of the present invention. The compositions of the present invention may contain a wide range of these additional, optional components. The total concentration of added ingredients usually is less than about 20%, preferably less than about 5%, and most preferably less than about 3% by weight of the total composition. Such components include, but are not limited to surfactants, emollients, moisturizers, stabilizers, film-forming substances, fragrances, colorants, chelating agents, preservatives, antioxidants, pH adjusting agents, antimicrobial agents, water-proofing agents, dry feel modifiers, vitamins, plant extracts, hydroxy acids (such as α-hydroxy acids and β-hydroxy acids), and sunless tanning agents. Examples of common raw materials and suitable adjuvants for an acne treatment composition are described by Beumer et al. supra and Robinson et al., supra.

In one embodiment the AA may comprise a compound having an antiacne functionality contained within a polymeric coating, commonly in the form of a microsphere. Exemplary polymeric microspheres include, but are not limited to microspheres of polystyrene, polymethylmethacrylate, polyvinyltoluene, styrene/butadiene copolymer, and latex. For use in the invention, the microspheres have a diameter of about 10 nanometers to about 2 microns. Suitable microspheres, that are functionalized to enable covalent attachment, are available from companies such as Bang Laboratories (Fishers, Ind.).

### Methods for Treating or Preventing Acne

In another embodiment, a method is provided for treating or preventing acne. Specifically, the present invention also provides a method for treating or preventing acne comprising applying a skin care composition comprising at least one peptide-based antiacne reagent, as described above, to the skin. The skin care composition may be rinsed from the skin or left on the skin, depending upon the type of composition used. The compositions of the present invention may be applied to the skin by various means, including, but not limited to spraying, brushing, and applying by hand.

### EXAMPLES

The present invention is further illustrated in the following Examples. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” or “hr” means hour(s), “μL” means microliter(s), “mL” means milliliter(s), “L” means liter(s), “nm” means nanometer(s), “mm” means millimeter(s), “cm” means centimeter(s), “μm” means micrometer(s) or micron(s), “mM” means millimolar, “M” means molar, “mmol” means millimole(s), “μmole” means micromole(s), “g” means gram(s), “pg” means microgram(s), “mg” means milligram(s), “g” means the gravitation constant, “rpm” means revolutions per minute, “qs” means as much as suffices, “wt %” means weight percent and “MALDI mass spectrometry” means matrix-assisted, laser desorption ionization mass spectrometry.

### Example 1 (Prophetic)

### Preparation of a Peptide-Based Antiacne Reagent

The purpose of this prophetic example is to describe how to prepare a peptide-based antiacne reagent by covalently coupling the antiacne agent salicylic acid to a skin-binding peptide.

Salicyloyl chloride (CAS No. 1441-87-8) is added dropwise at room temperature in equimolar proportions to a dry dimethylformamide (DMF) solution of a skin binding peptide with an added N-terminal lysine residue, given as SEQ ID NO:7. A hydrohalide acceptor such as triethylamine may be added in molar excess to catalyze formation of the amide bond between the peptide N terminus and 2-hydroxy benzoyl chloride. The resulting mixture is stirred at room temperature for several hours, after which time the product is isolated by removal of the solvent at reduced pressure. The product is further purified by organic extraction, liquid chromatography or dialysis. Adduct formation is confirmed by liquid chromatography/mass spectrometry. The purified product is then applied to skin in the presence of a suitable peptidase to release the active ingredient, salicylic acid.

### Example 2 (Prophetic)

### Preparation of a Peptide-Based Antiacne Reagent

The purpose of this prophetic example is to describe how to prepare a peptide-based antiacne reagent by covalently coupling the antiacne agent 3-phenoxy-1-propanol to a skin-binding peptide.

The chloroformate derivative of 3-phenoxy-1-propanol (CAS No. 6180-61-6) is prepared by reaction with excess phosgene in refluxing dioxane, tetrahydrofuran, toluene or other suitable solvent. The product is purified by removal of excess phosgene and the reaction solvent is removed by vacuum distillation. The product is purified by recrystalization and confirmed via proton NMR spectroscopy. The purified product is then added dropwise from solution to a dimethylformamide solution of a skin binding peptide containing at one N-terminal lysine, given as SEQ ID NO:7. Covalent coupling takes place after stirring for several hours at room temperature through carbamate formation via the N-terminal amine group. Optionally a tertiary amine such as triethylamine may be added to catalyze the coupling reaction. The product is isolated by removal of the solvent at reduced pressure and is further purified by organic extraction, liquid chromatography or dialysis. Adduct formation is confirmed by liquid chromatography/mass spectrometry. The purified product is then applied to the skin as part of an ointment or cream formulation.

### Example 3 (Prophetic)

### Preparation of a Peptide-Based Antiacne Reagent

The purpose of this prophetic Example is to describe how to prepare a peptide-based antiacne reagent that is covalently linked via an ester bond that will be more labile to release of the active ingredient through hydrolysis.

A suspension of glutamic acid in excess 2-phenoxy ethanol (CAS No. 122-99-6) is heated in the presence of concentrated hydrochloric acid over several hours to yield the gamma phenoxy ethyl glutamate. The product is purified by precipitation into acetone, collected by filtration and then recrystalized from hot water or organic solvent mixtures. The purified product is then suspended in dry tetrahydrofuran (THF) or dioxane and heated at reflux under nitrogen in the presence of a 2 to 5 molar excess of phosgene to produce the N-carboxyanhydride of gamma phenoxyethyl glutamate. The product is purified by removal of THF and excess phosgene and then recrystalized from ethyl acetate or THF/hexane mixtures to yield the white crystalline N-carboxyanhydride. The product is confirmed by proton NMR spectroscopy.

The purified product is then added dropwise from solution in dimethylformamide to a 0.1 to 1 molar ratio of skin binding peptide having an N-terminal lysine residue, given as SEQ ID NO:7, also dissolved in DMF. Addition of the N-carboxyanhydride occurs via amide bond formation at the peptide N-terminus with release of carbon dioxide. Depending on the starting molar ratio, additional gamma phenoxyethyl glutamate units can be added in a stepwise manner to the peptide N-terminus. The product is isolated by removal of the solvent at reduced pressure and further purified by organic extraction, liquid chromatography or dialysis. Adduct formation is confirmed by liquid chromatography/mass spectrometry or MALDI mass spectrometry. The purified product is then applied to the skin as part of an ointment or cream formulation.

### Example 4 (Prophetic)

### Preparation of an Antiacne Skin Lotion Comprising a Peptide-Based Antiacne Reagent

The purpose of this prophetic Example is to describe the preparation of an antiacne skin lotion composition comprising a peptide-based antiacne reagent.

The antiacne skin lotion composition is prepared using the ingredients listed in Table 1.

TABLE 1
Antiacne Skin Lotion Composition

| Ingredient | Wt % |
|---|---|
| Glyceryl stearate | 6.0 |
| Isopropyl myristate | 3.0 |
| Stearic acid | 2.0 |
| Peptide-based antiacne reagent from | 5 |
| Example 1 | |
| Peptide-based antiacne reagent from | 3 |
| Example 2 | |
| Ethyl alcohol | 10.0 |
| Propylene glycol | 3.0 |
| Triethanolamine | 1.0 |
| Fragrance, colorant, preservative | 1.0 |
| Water | qs to 100 |

The skin lotion composition is prepared by adding water and, heating to 65° C. and mixing until the ingredients are dissolved. Then the remaining ingredients are added, and the mixture is mixed until all the solids are dissolved. The pH is adjusted with citric acid as desired.

### Example 5 (Prophetic)

### Preparation of an Antiacne Cream Comprising a Peptide-Based Antiacne Reagent

The purpose of this prophetic Example is to describe the preparation of an antiacne cream composition comprising a peptide-based antiacne reagent.

The antiacne cream composition is prepared using the ingredients listed in Table 2.

TABLE 2
Antiacne Skin Cream Composition

| CFTA Names | Wt % |
|---|---|
| Cetearyl alcohol | 1.5 |
| Ceteareth-20 | 1.0 |
| Diisopropyl adipate | 1.5 |
| Cellulose | 2.8 |
| Peptide-based antiacne reagent from Example 3 | 3.6 |
| PEG-75 | 5.0 |
| Fragrance, colorant, preservative | 1.0 |
| Water | qs to 100 |

To 55 g of deionized water heated to 60° C., the first 4 ingredients are added serially with moderate agitation until completely dissolved. The bulk solution is then cooled to 35° C., and the remaining ingredients are added serially with moderate agitation.

What is claimed is:

1. A peptide-based antiacne reagent having the general structure
(SBPₘ)ₙ−(AA)_{y}, wherein
a) SBP is a skin-binding peptide;
b) AA is an antiacne agent;
c) m ranges from 1 to about 100;
d) n ranges from 1 to about 100;
e) y ranges from 1 to about 100; and optionally comprising a spacer.

2. The peptide-based antiacne reagent of claim 1, wherein the skin-binding peptide is from about 7 to about 35 amino acids in length.

3. The peptide-based antiacne reagent of claim 1, wherein the skin-binding peptide is generated combinatorially by a process selected from the group consisting of phage display, yeast display, ribosome display, mRNA-display, and bacterial display or generated empirically.

4. The peptide-based antiacne reagent of claim 1, wherein the skin-binding peptide is selected from the group consisting of SEQ ID NOs: 1, 2, 3, 4, 5, 6, 7, 8, 9, 10, 11, 12, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, and 58.

5. The peptide-based antiacne reagent of claim 1, wherein the skin-binding peptide further comprises a cysteine or lysine residue on at least one end of the peptide selected from the group consisting of:
a) the N-terminal end; and
b) the C-terminal end.

6. The peptide-based antiacne reagent of claim 1, wherein the skin-binding peptide further comprises a proline or aspartic acid residue on at least one end of the peptide selected from the group consisting of
a) the N-terminal end; and
b) the C-terminal end.

7. The peptide-based antiacne reagent of claim 1, wherein the antiacne agent is selected from the group consisting of: keratolytics, retinoids, tretinoin, adapalene, tazarotene, sulfur-containing D and L amino acids, derivatives and salts of sulfur-containing D and L amino acids, lipoic acid, sebostats, bile salts, antibiotics and antimicrobials selected from the group consisting of benzoyl peroxide, octopirox, tetracycline, 2,4,4′-trichloro-2′-hydroxy diphenyl ether, 3,4,4′-trichlorobanilide, azelaic acid, derivatives of azelaic acid, phenoxyethanol, phenoxypropanol, phenoxisopropanol, ethyl acetate, clindamycin, erythromycin, triclosan, chlorhexidine gluconate, nicotinamide, tea tree oil, rofecoxib, and meclocycline, and combinations of these.

8. The peptide-based antiacne reagent of claim 1, wherein the antiacne agent is encapsulated in a polymeric delivery vehicle.

9. The peptide-based antiacne reagent of claim 8, wherein the polymeric delivery vehicle is comprised of a material selected from the group consisting of polystyrene, polymethylmethacrylate, polyvinyltoluene, styrene/butadiene copolymer, and latex.

10. The peptide-based antiacne reagent of claim 1, wherein the antiacne agent is an antimicrobial peptide.

11. The peptide-based antiacne reagent of claim 10, wherein the antimicrobial peptide is selected from the group consisting of: lantibiotics, defensins, attacins, cecropins, magainins, melittins, histatins, brevinins, novispirins, and combinations thereof.

12. The peptide-based antiacne reagent of claim 10, wherein the antimicrobial peptide is selected from the group consisting of: SEQ ID NOs: 59-87.

13. The peptide-based antiacne reagent of claim 1, wherein the skin-binding peptide is identified by a process comprising the steps of:
(a) providing a combinatorial library of DNA associated peptides;
(b) contacting the library of (a) with a skin sample to form a reaction solution comprising DNA associated peptide-skin complexes;
(c) isolating the DNA associated peptide-skin complexes of (b);
(d) amplifying the DNA encoding the peptide portion of the DNA associated peptide-skin complexes of (c); and
(e) sequencing the amplified DNA of (d) encoding a skin-binding peptide, wherein the skin-binding peptide is identified.

14. The peptide-based antiacne reagent of claim 13, wherein after step (c):
i) the DNA associated peptide-skin complexes are contacted with an eluting agent whereby a portion of DNA associated peptides are eluted from the skin and a portion of the DNA associated peptides remain complexed; and
ii) the eluted or complexed DNA associated peptides of (i) are subjected to steps (d) and (e).

15. The peptide-based antiacne reagent of claim 13, wherein the DNA encoding the peptides is amplified by a process selected from the group consisting of:
a) amplifying DNA comprising a peptide coding region by polymerase chain reaction; and
b) infecting a host cell with a phage comprising DNA encoding the peptide and growing said host cell in a suitable growth medium.

16. The peptide-based antiacne reagent of claim 13, wherein the peptides encoded by the amplified DNA of step (d) are contacted with a fresh skin sample and steps (b) through (d) are repeated one or more times.

17. The peptide-based antiacne reagent of claim 1, wherein the spacer is a peptide comprising amino acids selected from the group consisting of proline, lysine, glycine, alanine, serine, and mixtures thereof.

18. The peptide-based antiacne reagent of claim 17, wherein the peptide spacer is from 1 to about 50 amino acids in length.

19. The peptide-based antiacne reagent of claim 17, wherein the peptide spacer comprises an amino acid sequence selected from the group consisting of SEQ ID NO:13, 14, 15, and 16.

20. The peptide-based antiacne reagent of claim 1, wherein the spacer is selected from the group consisting of ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, ethyl alkyl chain, propyl alkyl chain, hexyl alkyl chain, steryl alkyl chains, cetyl alkyl chains, and palmitoyl alkyl chains.

21. A skin care composition comprising an effective amount of the peptide-based antiacne reagent of claim.

22. The skin care composition according to claim 21, wherein the skin care composition is selected from the group consisting of: lotions, creams, gels, sticks, sprays, ointments, cleansing liquid washes, cleansing solid bars, pastes, foams, powders, shaving creams, and wipes and wherein skin care composition further comprises at least one cosmetic raw material or adjuvant selected from the group consisting of: hydrocarbons, esters, fatty alcohols, fatty acids, emulsifying agents, humectants, viscosity modifiers, silicone based materials, surfactants, emollients, moisturizers, stabilizers, film-forming substances, fragrances, colorants, chelating agents, preservatives, antioxidants, pH adjusting agents, antimicrobial agents, water-proofing agents, dry feel modifiers, vitamins, plant extracts, hydroxy acids, organic sunscreen agents, inorganic sunscreen agents, peptide-based inorganic sunscreen agents, and sunless tanning agents.

23. A method for treating or preventing acne comprising the steps of:
a) providing a skin care composition comprising a peptide-based antiacne reagent selected from the group consisting of:
(SBPₘ)ₙ−(AA)_{y}; and i)
[(SBP)ₓ−Sₘ]ₙ−(AA)_{y} ii)
wherein
1) SBP is a skin-binding peptide;
2) AA is an antiacne agent;
3) n ranges from 1 to about 100;
4) S is a spacer;
5) m ranges from 1 to about 100;
8) x ranges from 1 to about 10; and
9) y ranges from 1 to about 100;
and wherein the skin binding peptide is selected by a method comprising the steps of:
A) providing a combinatorial library DNA associated peptides;
B) contacting the library of (A) with a skin sample to form a reaction solution comprising DNA associated peptide-skin complexes;
C) isolating the DNA associated peptide-skin complexes of (B);
D) amplifying the DNA encoding the peptide portion of the DNA associated peptide-skin complexes of (C); and
E) sequencing the amplified DNA of (d) encoding a skin-binding peptide, wherein the skin-binding peptide is identified; and
b) applying the skin care composition of (a) to the skin.

24. The method of claim 23, wherein after step (C):
i) the DNA associated peptide-skin complexes are contacted with an eluting agent whereby a portion of DNA associated peptides are eluted from the skin and a portion of the DNA associated peptides remain complexed; and
ii) the eluted or complexed DNA associated peptides of (i) are subjected to steps (D) and (E); and
wherein the DNA encoding the peptides is amplified by a process selected from the group consisting of:
amplifying DNA comprising a peptide coding region by polymerase chain reaction; and
infecting a host cell with a phage comprising DNA encoding the peptide and growing said host cell in a suitable growth medium.

25. The method of claim 23, wherein the peptides encoded by the amplified DNA of step (D) are contacted with a fresh skin sample and steps (B) through (D) are repeated one or more times.


SEQUENCE LISTING (87 sequences)

SEQ ID NO: 1 - protein, 12 aa, Artificial Sequence
  Skin-Binding Peptide
TPFHSPENAPGS

SEQ ID NO: 2 - protein, 12 aa, Artificial Sequence
  Skin-Binding Peptide
KRGRHKRPKRHK

SEQ ID NO: 3 - protein, 7 aa, Artificial Sequence
  Skin-Binding Peptide
RLLRLLR

SEQ ID NO: 4 - protein, 12 aa, Artificial Sequence
  Skin-Binding Peptide
HKPRGGRKKALH

SEQ ID NO: 5 - protein, 18 aa, Artificial Sequence
  Skin-Binding Peptide
KPRPPHGKKHRPKHRPKK

SEQ ID NO: 6 - protein, 18 aa, Artificial Sequence
  Skin-Binding Peptide
RGRPKKGHGKRPGHRARK

SEQ ID NO: 7 - protein, 12 aa, Artificial Sequence
  Skin-Binding Peptide
KQATFPPNPTAY

SEQ ID NO: 8 - protein, 12 aa, Artificial Sequence
  Skin-Binding Peptide
HGHMVSTSQLSI

SEQ ID NO: 9 - protein, 7 aa, Artificial Sequence
  Skin-Binding Peptide
LSPSRMK

SEQ ID NO: 10 - protein, 7 aa, Artificial Sequence
  Skin-Binding Peptide
LPIPRMK

SEQ ID NO: 11 - protein, 7 aa, Artificial Sequence
  Skin-Binding Peptide
HQRPYLT

SEQ ID NO: 12 - protein, 7 aa, Artificial Sequence
  Skin-Binding Peptide
FPPLLRL

SEQ ID NO: 13 - protein, 8 aa, Artificial Sequence
  Caspase 3 Cleavage Site
LESGDEVD

SEQ ID NO: 14 - protein, 37 aa, Artificial Sequence
  Peptide Spacer
TSTSKASTTTTSSKTTTTSSKTTTTTSKTSTTSSSST

SEQ ID NO: 15 - protein, 22 aa, Artificial Sequence
  Peptide Spacer 2
GQGGYGGLGSQGAGRGGLGGQG

SEQ ID NO: 16 - protein, 10 aa, Artificial Sequence
  Peptide Spacer
GPGGYGPGQQ

SEQ ID NO: 17 - protein, 13 aa, Artificial Sequence
  Skin-Binding Peptide
TPFHSPENAPGSK

SEQ ID NO: 18 - protein, 12 aa, Artificial Sequence
  Skin care composition-resistant skin-binding peptide
TMGFTAPRFPHY

SEQ ID NO: 19 - protein, 12 aa, Artificial Sequence
  Skin care composition-resistant skin-binding peptide
SVSVGMKPSPRP

SEQ ID NO: 20 - protein, 15 aa, Artificial Sequence
  Skin care composition-resistant skin-binding peptide
QLSYHAYPQANHHAP

SEQ ID NO: 21 - protein, 14 aa, Artificial Sequence
  Skin care composition-resistant skin-binding peptide
SGCHLVYDNGFCDH

SEQ ID NO: 22 - protein, 13 aa, Artificial Sequence
  Skin care composition-resistant skin-binding peptide
KTMGFTAPRFPHY

SEQ ID NO: 23 - protein, 16 aa, Artificial Sequence
  Synthetic peptides
TPFHSPENAPGSGGGS

SEQ ID NO: 24 - protein, 17 aa, Artificial Sequence
  Synthetic peptides
TPFHSPENAPGSGGGSS

SEQ ID NO: 25 - protein, 15 aa, Artificial Sequence
  Synthetic peptides
TPFHSPENAPGSGGG

SEQ ID NO: 26 - protein, 7 aa, Artificial Sequence
  Synthetic peptides
FTQSLPR

SEQ ID NO: 27 - protein, 7 aa, Artificial Sequence
  Synthetic peptides
QATFMYN

SEQ ID NO: 28 - protein, 11 aa, Artificial Sequence
  Synthetic peptides
VLTSQLPNHSM

SEQ ID NO: 29 - protein, 7 aa, Artificial Sequence
  Synthetic peptides
HSTAYLT

SEQ ID NO: 30 - protein, 12 aa, Artificial Sequence
  Synthetic peptides
APQQRPMKTFNT

SEQ ID NO: 31 - protein, 12 aa, Artificial Sequence
  Synthetic peptides
APQQRPMKTVQY

SEQ ID NO: 32 - protein, 7 aa, Artificial Sequence
  Synthetic peptides
PPWLDLL

SEQ ID NO: 33 - protein, 7 aa, Artificial Sequence
  Synthetic peptides
PPWTFPL

SEQ ID NO: 34 - protein, 7 aa, Artificial Sequence
  Synthetic peptides
SVTHLTS

SEQ ID NO: 35 - protein, 7 aa, Artificial Sequence
  Synthetic peptides
VITRLTS

SEQ ID NO: 36 - protein, 12 aa, Artificial Sequence
  Synthetic peptides
DLKPPLLALSKV

SEQ ID NO: 37 - protein, 12 aa, Artificial Sequence
  Synthetic peptides
SHPSGALQEGTF

SEQ ID NO: 38 - protein, 12 aa, Artificial Sequence
  Synthetic peptides
FPLTSKPSGACT

SEQ ID NO: 39 - protein, 12 aa, Artificial Sequence
  Synthetic peptides
DLKPPLLALSKV

SEQ ID NO: 40 - protein, 7 aa, Artificial Sequence
  Synthetic peptides
PLLALHS

SEQ ID NO: 41 - protein, 7 aa, Artificial Sequence
  Synthetic peptides
VPISTQI

SEQ ID NO: 42 - protein, 12 aa, Artificial Sequence
  Synthetic peptides
YAKQHYPISTFK

SEQ ID NO: 43 - protein, 7 aa, Artificial Sequence
  Synthetic peptides
HSTAYLT

SEQ ID NO: 44 - protein, 12 aa, Artificial Sequence
  Synthetic peptides
STAYLVAMSAAP

SEQ ID NO: 45 - protein, 12 aa, Artificial Sequence
  Synthetic peptides
NLQHSVGTSPVW

SEQ ID NO: 46 - protein, 12 aa, Artificial Sequence
  Synthetic peptides
NQAASITKRVPY

SEQ ID NO: 47 - protein, 14 aa, Artificial Sequence
  Synthetic peptides
ASCPSASHADPCAH

SEQ ID NO: 48 - protein, 14 aa, Artificial Sequence
  Synthetic peptides
NLCDSARDSPRCKV

SEQ ID NO: 49 - protein, 12 aa, Artificial Sequence
  Synthetic peptides
NHSNWKTAADFL

SEQ ID NO: 50 - protein, 12 aa, Artificial Sequence
  Synthetic peptides
GSSTVGRPLSYE

SEQ ID NO: 51 - protein, 12 aa, Artificial Sequence
  Synthetic peptides
SDTISRLHVSMT

SEQ ID NO: 52 - protein, 12 aa, Artificial Sequence
  Synthetic peptides
SPLTVPYERKLL

SEQ ID NO: 53 - protein, 12 aa, Artificial Sequence
  Synthetic peptides
SPYPSWSTPAGR

SEQ ID NO: 54 - protein, 12 aa, Artificial Sequence
  Synthetic peptides
VQPITNTRYEGG

SEQ ID NO: 55 - protein, 12 aa, Artificial Sequence
  Synthetic peptides
WPMHPEKGSRWS

SEQ ID NO: 56 - protein, 14 aa, Artificial Sequence
  Synthetic peptides
DACSGNGHPNNCDR

SEQ ID NO: 57 - protein, 14 aa, Artificial Sequence
  Synthetic peptides
DHCLGRQLQPVCYP

SEQ ID NO: 58 - protein, 14 aa, Artificial Sequence
  Synthetic peptides
DWCDTIIPGRTCHG

SEQ ID NO: 59 - protein, 17 aa, Artificial Sequence
  synthetic construct
PKGLKKLLKGLKKLLKL

SEQ ID NO: 60 - protein, 16 aa, Artificial Sequence
  synthetic construct
KGLKKLLKGLKKLLKL

SEQ ID NO: 61 - protein, 16 aa, Artificial Sequence
  synthetic construct
KGLKKLLKLLKKLLKL

SEQ ID NO: 62 - protein, 14 aa, Artificial Sequence
  synthetic construct
LKKLLKLLKKLLKL

SEQ ID NO: 63 - protein, 12 aa, Artificial Sequence
  synthetic construct
LKKLLKLLKKLL

SEQ ID NO: 64 - protein, 17 aa, Artificial Sequence
  synthetic construct
VAKKLAKLAKKLAKLAL

SEQ ID NO: 65 - protein, 13 aa, Artificial Sequence
  synthetic construct
FAKLLAKALKKLL

SEQ ID NO: 66 - protein, 16 aa, Artificial Sequence
  synthetic construct
KGLKKGLKLLKKLLKL

SEQ ID NO: 67 - protein, 16 aa, Artificial Sequence
  synthetic construct
KGLKKLLKLGKKLLKL

SEQ ID NO: 68 - protein, 16 aa, Artificial Sequence
  synthetic construct
KGLKKLGKLLKKLLKL

SEQ ID NO: 69 - protein, 16 aa, Artificial Sequence
  synthetic construct
KGLKKLLKLLKKGLKL

SEQ ID NO: 70 - protein, 16 aa, Artificial Sequence
  synthetic construct
KGLKKLLKLLKKLGKL

SEQ ID NO: 71 - protein, 19 aa, Artificial Sequence
  synthetic construct
FALALKALKKLKKALKKAL

SEQ ID NO: 72 - protein, 17 aa, Artificial Sequence
  synthetic construct
FAKKLAKLAKKLAKLAL

SEQ ID NO: 73 - protein, 13 aa, Artificial Sequence
  synthetic construct
FAKLLAKLAKKLL

SEQ ID NO: 74 - protein, 15 aa, Artificial Sequence
  synthetic construct
FAKKLAKLALKLAKL

SEQ ID NO: 75 - protein, 10 aa, Artificial Sequence
  synthetic construct
FAKKLAKKLL

SEQ ID NO: 76 - protein, 13 aa, Artificial Sequence
  synthetic construct
FAKLLAKLAKKVL

SEQ ID NO: 77 - protein, 13 aa, Artificial Sequence
  synthetic construct
KYKKALKKLAKLL

SEQ ID NO: 78 - protein, 12 aa, Artificial Sequence
  synthetic construct
FALLKALLKKAL

SEQ ID NO: 79 - protein, 14 aa, Artificial Sequence
  synthetic construct
KRLFKKLKFSLRKY

SEQ ID NO: 80 - protein, 14 aa, Artificial Sequence
  synthetic construct
KRLFKKLLFSLRKY

SEQ ID NO: 81 - protein, 14 aa, Artificial Sequence
  synthetic construct
LLLFLLKKRKKRKY

SEQ ID NO: 82 - protein, 36 aa, H. cecropia
KWKLFKKIEKVGQNIRDGIIKAGPAVAWGQATQIAK

SEQ ID NO: 83 - protein, 23 aa, Xenopus
GIGKFLHSAKKFGKAFVGEIMNS

SEQ ID NO: 84 - protein, 22 aa, Xenopus
GIGKFLKKAKKFGKAFVKILKK

SEQ ID NO: 85 - protein, 12 aa, Bos taurus
RLCRIVVIRVCR

SEQ ID NO: 86 - protein, 13 aa, Bos sp.
ILPWKWPWWPWRR

SEQ ID NO: 87 - protein, 24 aa, Homo sapiens
DSHAKRHHGYKRKFHEKHHSHRGY